(19) United States
(12) Patent Application Publication
(10) Pub. No.: US 2021/0382054 A1
(43) Pub. Date: Dec. 9, 2021

(54) METHODS FOR SCREENING UBIQUITIN LIGASE AGONISTS

(71) Applicant: University of Washington, Seattle, WA (US)

(72) Inventors: Ning Zheng, Seattle, WA (US); Shiyun Cao, Seattle, WA (US); Thomas R. Hinds, Seattle, WA (US); Heng Li, Seattle, WA (US); Haibin Mao, Seattle, WA (US)

(73) Assignee: University of Washington, Seattle, WA (US)

(21) Appl. No.: 17/283,773

(22) PCT Filed: Oct. 17, 2019
(86) PCT No.: PCT/US2019/056738

§ 371 Date: Apr. 8, 2021

Related U.S. Application Data
(60) Provisional application No. 62/746,957, filed on Oct. 17, 2018.

(51) Int. Cl. G01N 33/573; C12N 9/00; C12Q 1/25; G01N 33/53; G01N 33/566

(52) U.S. Cl. CPC G01N 33/573; C12N 9/93; C12Q 1/25; G01N 21/6428; G01N 33/566; C12Y 603/02019; G01N 2333/9015; G01N 33/5306

(57) ABSTRACT

Disclosed herein are methods for identifying a ubiquitin ligase agonist, and the methods include (a) contacting a ubiquitin ligase with a candidate agonist and a neo-substrate; and (b) determining whether the candidate agonist is effective to result in binding the ubiquitin ligase to the neo-substrate, wherein binding of the ubiquitin substrate to the neo-substrate identifies the candidate agonist as a ubiquitin ligase agonist.

### CROSS-REFERENCE TO RELATED APPLICATION

This application claims the benefit of U.S. Application No. 62/746,957, filed Oct. 17, 2018, expressly incorporated herein by reference in its entirety.

### STATEMENT REGARDING SEQUENCE LISTING

The sequence listing associated with this application is provided in text format in lieu of a paper copy and is hereby incorporated by reference into the specification. The name of the text file containing the sequence listing is 70157_Sequence_final_2019-10-16.txt. The text file is 2 KB; was created on Oct. 16, 2019; and is being submitted via EFS-Web with the filing of the specification.

### BACKGROUND OF THE INVENTION

The ubiquitin-proteasome system (UPS) regulates diverse cellular functions by mediating the turnover of a myriad of proteins in eukaryotic cells (Hershko, A. and Ciechanover, A. The ubiquitin system. Annu Rev Biochem 67, 425-479, doi: 10.1146/annurev.biochem.67.1.425 (1998)). To tag a protein for degradation, eukaryotic cells first covalently modify the protein by a poly-ubiquitin chain, which serves as a proteasome-targeting signal. This post-translational modification (i.e. ubiquitination) is achieved by the sequential actions of three classes of enzymes, E1, E2, and E3 (Pickart, C. M. Mechanisms underlying ubiquitination. Annu Rev Biochem 70, 503-533, doi:10.1146/annurev.biochem.70.1.503 (2001)). Among these enzymes, ubiquitin E3 ligases play a central role in the enzymatic cascade by recognizing a specific protein substrate and promoting the transfer of ubiquitin from the ubiquitin-conjugating E2 enzyme to the target protein (Zheng, N. & Shabek, N. Ubiquitin Ligases: Structure, Function, and Regulation. Annu Rev Biochem 86, 129-157, doi:10.1146/annurev-biochem-060815-014922 (2017)). The specificity of ubiquitination is, therefore, dictated by the E3-substrate interface. Humans have hundreds of different E3s. With the help of additional adaptor proteins, these E3 ligases are able to recognize and ubiquitinate hundreds, if not thousands, of substrate proteins with high specificity. Nonetheless, there exists proteins that are not substrates to E3 ligases.

There is a need to establish effective methods for discovering and developing molecular glue E3 agonists that can rewire ubiquitin ligases to ubiquitinate and degrade desirable substrates that otherwise are not processed by the E3s. The present invention seeks to fulfill this need and provide further related advantages.

### SUMMARY OF THE INVENTION

In one aspect, a method for identifying a ubiquitin ligase agonist is disclosed. In one embodiment, the method comprises (a) contacting a ubiquitin ligase with a candidate agonist and a neo-substrate; and (b) determining whether the candidate agonist is effective to result in binding the ubiquitin ligase to the neo-substrate, wherein binding of the ubiquitin substrate to the neo-substrate identifies the candidate agonist as a ubiquitin ligase agonist. In certain embodiments, binding of the ubiquitin ligase to the neo-substrate provides a complex comprising the ubiquitin ligase, the agonist, and the neo-substrate.

In certain embodiments of the method, determining whether the candidate agonist is effective to result in binding the ubiquitin ligase to the neo-substrate comprises observing a signal generated by the binding.

In certain embodiments of the method, the ubiquitin ligase further comprises a first reporting agent and the substrate further comprises a second reporting agent, wherein upon binding of the ubiquitin ligase to the neo-substrate, the first reporting agent acts with the second reporting agent to generate a signal. In certain of these embodiments, the ubiquitin ligase comprising the first reporting agent is a donor bead configured to generate a reactive oxygen species when in the excited state, and the neo-substrate comprising the second reporting agent is an acceptor bead configured to generate light in the presence of a reactive oxygen species upon binding of the ubiquitin ligase to the neo-substrate. In certain of these embodiments, the donor bead comprises a sensitizer configured to generate the reactive oxygen species when the sensitizer is in an excited state. In certain of these embodiments, the sensitizer is a photosensitizer configured to generate the reactive oxygen species when the sensitizer is illuminated with stimulation electromagnetic radiation. In certain embodiments, the photosensitizer is a phthalocyanine. In certain embodiments, the acceptor bead comprises a luminescent compound configured to generate luminescent light when the luminescent compound is in proximity to a reactive oxygen species. Representative luminescent compounds include thioxene, anthracene, rubrenein, and combinations thereof. In certain embodiments, the reactive oxygen species is singlet oxygen.

In certain embodiments of the method, determining whether the candidate agonist is effective to result in binding the ubiquitin ligase to the neo-substrate comprises observing a gain of a signal by the binding.

In certain embodiments of the method, the ubiquitin ligase further comprises a first reporting agent and the neo-substrate further comprises a second reporting agent, wherein upon binding of the ubiquitin ligase to the neo-substrate, the first reporting agent acts with the second reporting agent resulting in a gain of a signal.

In certain embodiments of the method, the ubiquitin ligase is a member of the cullin-RING superfamily of multi-subunit E3 ubiquitin ligases, a member of RING-type E3 ligases with a substrate binding domain, or a member of HECT-type E3 ligases with a substrate binding domain.

In certain embodiments of the method, the ubiquitin ligase is KEAP1.

In certain embodiments of the method, the neo-substrate is KRAS or KRAS mutant.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1. A comparison between PROTACs and Molecular Glues as E3 agonists.

FIG. 2. Schematic drawing of a molecular glue compound capable of promoting interactions between KEAP1 and KRAS.

FIG. 3. The design of the AlphaScreen-based primary screen and counter-screen assays as well as secondary screen assays originally planned. His-tagged KRAS is immobilized to the acceptor bead, whereas biotinylated KEAP1 is bound to the donor bead. If a small molecule compound can promote KEAP1-KRAS interaction, a signal will be produced in AlphaScreen. An RBD-NRF2 degron fusion protein will be used as a positive control.

FIG. 4. Cisbio TR-FRET as a secondary screen. Schematic drawing of the principle behind TR-FRET assay, which is also a proximity-based protein-protein interaction assay.

FIG. 5. Design of the adjustable platform for tracking low affinity binding (APTLAB) assay. This assay was developed for detecting weak interactions between two proteins that are induced by a molecule glue compound. It uses adjustable weak interactions between DNA oligos to enhance the compound-induced weak protein-protein interactions. In this system, the two proteins are individually conjugated with a single-stranded DNA, which can be brought together by a longer piece of single stranded DNA with sequences complementary to the protein-conjugated DNA oligos. The DNA-protein conjugation is mediated by a bacterial HUH protein, which is fused to each individual protein and can catalyze its conjugation to a specific DNA sequence through a tyrosine residue.

FIGS. 6A and 6B. DNA oligos used for APTLAB. In addition to the HUH-specific DNA sequence (ACCAG), the DNA oligos feature different lengths with sequences complementary to the bridge oligo. This adjustable feature can be used to introduce graded affinity between the DNA oligos.

FIGS. 7A and 7B. Conjugation of single stranded DNA oligos to KEAP1 and KRAS. SDS-PAGE analysis of KEAP-HUH and KRAS-HUH before and after being conjugated to two DNA oligos with different length.

FIGS. 8A-8C. Design of three ssDNA bridge oligos with different lengths. (A) The specific sequence of the three oligos. (B) The activity of the three oligos in producing binding signal detected by Octet BLI. (C) The effect of one hit compound on the binding signal detected by Octet BLI in the presence of two oligos.

FIG. 9. Summary of the activities of 19 hit compounds validated by APTLAB. The Octet BLI binding signal at 10 minutes after binding was initiated was plotted on Y-axis.

FIG. 10. Design of the split luciferase assay for hit validation. Compound-induced weak interactions between KEAP1 and KRAS can promote the interactions between two halves of luciferase individually fused to one of the binding proteins. The activity of the hit compounds can be detected through enhanced luciferase activity.

FIG. 11. Summary of hit compound activities validated by the split luciferase assay.

### DETAILED DESCRIPTION OF THE INVENTION

Some embodiments relate to methods for the discovery of small molecule compounds (e.g., molecular glues) that foster interactions between a non-druggable oncogene product and a human E3 ligase that otherwise does not bind the oncogene product (i.e., through the action of the molecular glue becomes a neo-substrate of the ligase).

Some embodiments relate to methods for screening a ubiquitin ligase agonist, comprising: contacting a ubiquitin ligase with a candidate agonist and a neo-substrate; and measuring a binding activity of the ubiquitin ligase to the neo-substrate in the presence of the candidate agonist.

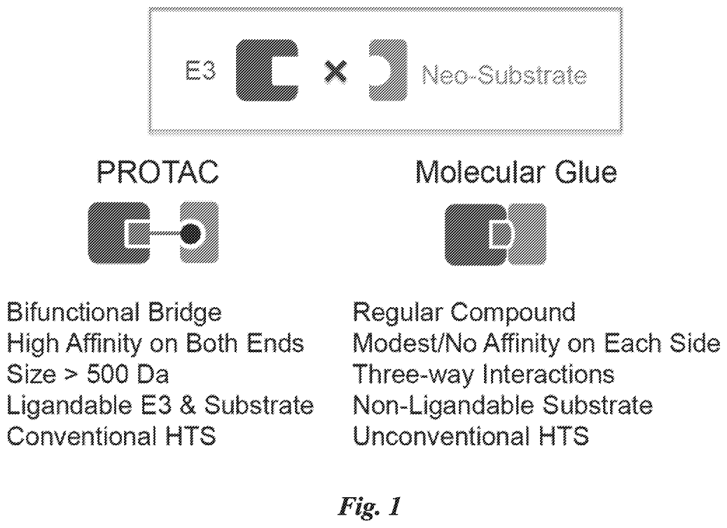

As used herein the term “neo-substrate” refers to a protein that is not naturally a substrate for a given ubiquitin ligase. The term “neo-substrate” refers to a protein that binds to a given ubiquitin ligase only in the presence of an agonist that serves as a molecular glue that is effective to provide a complex comprising the ligase, the agonist, and the neo-substrate. In contrast to PROTAC compounds (protein-targeting chimeric molecules), molecular glues have only modest or no affinity to each protein, but rather promote three-way interactions (FIG. 1). Although both PROTACs and molecular glues can be E3 agonists and promote targeted protein degradation, they are mechanistically distinct. PROTACs are bifunctioinal molecules with two separate warheads connected by a linker. These warheads need to have high enough affinity to recruit the E3 and the substrate simultaneously. This requirement makes PROTACs intrinsically large, readily exceeding 500 Da in molecular weight. The substrates of PROTACs have to be ligandable proteins in order to show affinity toward a warhead chemical moiety. The approach of developing PROTACs involves discovering compounds that can separately bind E3 and substrates using conventional high-throughput screen methods and connecting the two compounds together with a linker moiety. By contract, molecular glue compounds do not have to show high affinity toward substrates, which can be non-ligandable targets. The expected molecular weight of molecule glue compounds will be in the same range as typical small molecules.

In some embodiments, the candidate agonist has a molecular weight of less than 500 Da, less than 250 D, less than 200 Da, less than 175 Da, less than 125 Da, or less than 100 Da. In some embodiments, the candidate agonist has substantially no affinity to the neo-substrate. In some embodiments, the candidate agonist has no affinity to the neo-substrate. In some embodiments, the candidate agonist has substantially no affinity to the ubiquitin ligase. In some embodiments, the candidate agonist has no affinity to the ubiquitin ligase. In some embodiments, the candidate agonist does not comprise both a moiety that binds with the neo-substrate and a moiety that binds with the ubiquitin ligase (e.g., it is not bi-functional).

In some embodiments, the dose response of the candidate agonist in the presence of the ubiquitin ligase and the neo-substrate does not exhibit a substantial decrease in ligase/neo-substrate binding upon increasing concentration of the candidate agonist. In some embodiments, the degree of ligase/neo-substrate binding does not decrease more than 5%, 10%, 15%, 25%, 30%, 35%, 40%, 45%, or 50% of the maximum degree of binding at concentrations higher than the concentration at maximum ligase/neo-substrate binding.

In some embodiments, the method described herein includes measuring the binding activity using one or more assays. In some embodiments, the screening method includes measuring the binding activity using a primary assay and measuring the binding activity using one or more secondary assays for validation.

In some embodiments, the assay comprises the ubiquitin ligase and the neo-substrate.

In some embodiments, the primary assay is an amplified luminescent proximity homogenous assay. In some embodiments, the secondary assay is selected from TR-FRET binding assay, Biacore binding assay, Octet BLI binding assay, or in vitro activity assay.

In some embodiments, binding of the ubiquitin ligase to the neo-substrate provides a complex comprising the ubiquitin ligase, the agonist, and the neo-substrate.

In some embodiments, the method described herein includes screening a candidate agonist to determine whether the candidate agonist is effective to result in binding the ubiquitin ligase to the neo-substrate. In some embodiments, the step of screening the candidate agonist comprises observing a signal generated by the binding.

In some embodiments, the ubiquitin ligase further comprises a first reporting agent and the substrate further comprises a second reporting agent, wherein upon binding of the ubiquitin ligase to the neo-substrate, the first reporting agent acts with the second reporting agent to generate a signal.

In some embodiments, the ubiquitin ligase comprising the first reporting agent is a donor bead configured to generate a reactive oxygen species when in the excited state, and the neo-substrate comprising the second reporting agent is an acceptor bead configured to generate light in the presence of a reactive oxygen species upon binding of the ubiquitin ligase to the neo-substrate.

In some embodiments, the donor bead comprises a sensitizer configured to generate the reactive oxygen species when the sensitizer is in an excited state.

In some embodiments, the sensitizer is a photosensitizer configured to generate the reactive oxygen species when the sensitizer is illuminated with stimulation electromagnetic radiation.

In some embodiments, the photosensitizer is a phthalocyanine.

In some embodiments, wherein the acceptor bead comprises a luminescent compound configured to generate luminescent light when the luminescent compound is in proximity to a reactive oxygen species.

In some embodiments, the luminescent compound is selected from the group consisting of thioxene, anthracene, rubrenein, and combinations thereof.

In some embodiments, the reactive oxygen species is singlet oxygen.

In some embodiments, determining whether the candidate agonist is effective to result in binding the ubiquitin ligase to the neo-substrate comprises observing a gain of a signal by the binding.

In some embodiments, the ubiquitin ligase further comprises a first reporting agent and the neo-substrate further comprises a second reporting agent, wherein upon binding of the ubiquitin ligase to the neo-substrate, the first reporting agent acts with the second reporting agent resulting in a gain of a signal.

In some embodiments, the ubiquitin ligase is a member of the cullin-RING superfamily of multi-subunit E3 ubiquitin ligases, a member of RING-type E3 ligases with a substrate binding domain, or a member of HECT-type E3 ligases with a substrate binding domain. In some embodiments, the ubiquitin ligase is a member of the HECT, RING-type, U-box, and PHD-finger type of ligase with a substrate binding domain.

In some embodiments, the ubiquitin ligase is KEAP1. In some embodiments, the neo-substrate is KRAS or KRAS mutant.

Methods of Screening

The following describes a representative method for screening small molecules to identify small molecule agonists of a ubiquitin ligase for targeted neo-substrate degradation (via ubiquitination).

In some embodiments, the assay used in the method described herein are designed for screening the ubiquitin ligase binding activity. In some embodiments, the assay used in the method described herein is for screening KEAP1 and KRAS/KRAS mutant binding.

In some embodiments, two or more assays are used for screening. In some embodiments, the screening method comprises screening candidate agonists with a primary assay. In some embodiments, the screening method comprises screening candidate agonists with a secondary screen assay for validating the binding activity.

In some embodiments, the assay used in the screening is an amplified luminescent proximity homogenous assay, wherein the assay comprises a ubiquitin ligase and a neo-substrate. In some embodiments, the assay comprises a ubiquitin ligase attached a donor bead and a neo-substrate attached to a receptor bead. In some embodiments, the assay comprises a ubiquitin ligase attached an acceptor bead and a neo-substrate attached to a donor bead. In some embodiments, when the ubiquitin binds to the neo-substrate in the presence of the ubiquitin ligase agonist, the donor bead and the accept bead interacts to generate a signal.

In some embodiments, the assay used for screening is an adjustable platform for tracking low affinity binding assay (APTLAB). In some embodiments, the assay used in the screening is a TR-FRET (time resolved-fluorescence resonance energy transfer). In some embodiments, the assay used in the screening is split luciferase assay. In some embodiments, the method described herein includes measuring the binding activity using one or more assays. In some embodiments, the screening method includes measuring the binding activity using a primary assay and measuring the binding activity using one or more secondary assays for validation.

In some embodiments, the assay comprises the ubiquitin ligase and the neo-substrate.

In some embodiments, the primary assay is an amplified luminescent proximity homogenous assay. In some embodiments, the assay utilizes the adjustable interactions between DNA oligos that can be used to enhance the binding between the ubiquitin ligase and the neo-substrate in the presence of the ubiquitin ligase agonist. In some embodiments, the ubiquitin ligase is conjugated to a first single-stranded DNA and the neo-substrate is conjugated with a second single-stranded DNA, wherein the first single-stranded DNA and the second single-stranded DNA are brought together by a longer piece of single stranded DNA with sequences complementary to the protein-conjugated first and second single-stranded DNAs. The DNA-protein conjugation is mediated by a bacterial HUH protein, which is fused to each individual protein and can catalyze its conjugation to a specific DNA sequence through a tyrosine residue. In some embodiments, the binding of the ubiquitin ligase and the neo-substrate in the presence of the ubiquitin ligase agonist can be enhanced through the interaction between the complementary DNA and result in a detectable signal.

In some embodiments, the secondary assay is selected from TR-FRET binding assay, Biacore binding assay, Octet BLI binding assay, or in vitro activity assay.

Target Selection

In some embodiments, the method described herein comprises first selecting a neo-substrate. In some embodiments, the method described herein comprises selecting a substrate protein. In some embodiments, the substrate protein can be ubiquitinated and degraded. In some embodiments, the method described herein comprises selecting one or more E3 ubiquitin ligases that can ubiquitinate the substrate protein upon binding to the neo-substrate and substrate protein. In some embodiments, the substrate protein and E3 ubiquitin ligase can bind together after interacting with the neo-substrate. Unlike conventional drug discovery, the effort to chemically reprogram an E3 ligase to ubiquitinate a neo-substrate entails two “targets”, the ubiquitin E3 ligase and the substrate protein. As a first step, a “substrate-centric” approach was taken by first selecting the protein of interest to be ubiquitinated and degraded. This is in contrast to a “ligase-centric” approach, which focuses on a specific E3 and explores its potential to ubiquitinate different substrates. Once a substrate is identified, one or more appropriate E3s were chosen based on several criteria specified below.

In some embodiments, the neo-substrate protein does not bind directly to E3 ubiquitin ligase in the absence of a neo-substrate. A number of human diseases are driven by mutated, dysregulated, or deleterious gene products, whose down-regulation can slow down disease progression and alleviate disease symptoms. To take advantage of the unique power of molecular glue E3 agonists, non-druggable and particularly non-ligand-able targets were considered. These targets could have a non-druggable globular domain or are intrinsically disordered without a ligand-able site. Mutations of KRAS and other RAS isoforms, especially at codon 12 and 61 can render the GTPase oncogenic, and are frequently found in pancreatic, lung, and colon cancers. Because of the GTP-binding pocket, KRAS mutants can be non-druggable due to the high affinity and high concentration of cellular GTP. Outside its nucleotide-binding pocket, KRAS present few deep surface cavity that can be targeted by small molecule. The highest affinity reported for existing KRAS-binding compound is at ˜100 μM (Maurer, T. et al. Small-molecule ligands bind to a distinct pocket in Ras and inhibit SOS-mediated nucleotide exchange activity. Proc Natl Acad Sci USA 109, 5299-5304, doi:10.1073/pnas.1116510109 (2012)). The oncogenic product, therefore, can be a target for downregulation by a molecular glue E3 agonist.

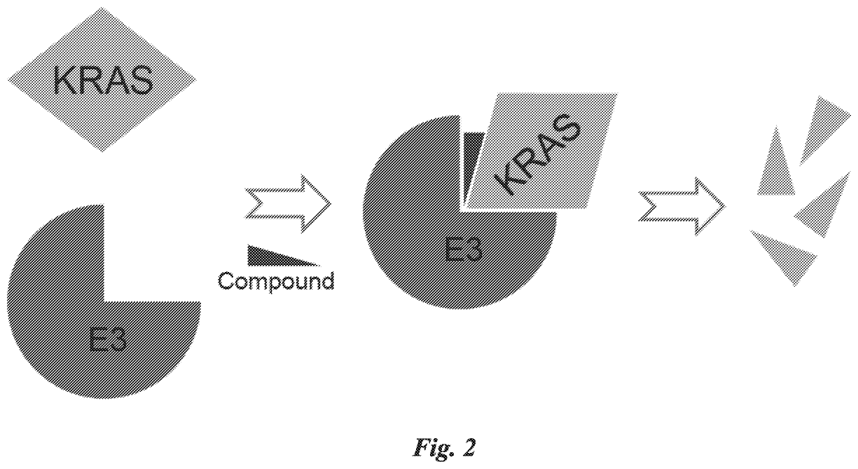

In some embodiments, the method described herein includes selecting a ubiquitin ligase that can bind and ubiquitinate KRAS or KRAS mutant. Several criteria can be used to select the appropriate E3: (1) the ubiquitin ligase is localized to the cytosol or plasma membrane, where KRAS is synthesized and functionalized; (2) the ubiquitin ligase is universally expressed in multiple tissues so that a working compound can be tested for different cancer indications; (3) the ubiquitin ligase is well characterized and known to promote substrate polyubiquitination and degradation instead of monoubiquitination or polyubiquitin chain assembly with non-Lys48 linkages; (4) the ubiquitin ligase is reasonably abundant so that its endogenous function is unlikely to be compromised by the molecular glue compound; and (5) the ubiquitin ligase has been structurally analyzed and is amendable for large scale purification. Among the E3 ligases, KEAP1 was identified as a candidate that satisfy most, if not all, criteria listed above (FIG. 2).

In some embodiments, the ubiquitin ligase is localized to the cytosol or plasma membrane. In some embodiments, the ubiquitin ligase is localized to where the neo-substrate is functionalized and synthesized. In some embodiments, the ubiquitin ligase is expressed in one or more tissues. In some embodiments, the ubiquitin ligase can promote substrate polyubiquitination and degradation instead of monoubiquitination or polyubiquitin chain assembly with non-Lys48 linkages. In some embodiments, the ubiquitin ligase is present in an amount sufficient to maintain its endogenous function after binding to the small molecule antagonist. In some embodiments, the ubiquitin ligase is suitable for large scale purification.

In some embodiments, the method described herein includes identifying neo-substrate that does not directly or naturally bind to the ubiquitin ligase.

In some embodiments, the method described herein includes screening compounds that foster the ubiquitin ligase and neo-substrate interaction. In some embodiments, the method described herein includes screening compounds that foster KEAP1 and KRAS interaction. In some embodiments, the method described herein includes screening the candidate agonist using a modified protein-protein interaction (PPI) assay. To identify compounds that are able to foster KRAS-KEAP1 interaction, small molecule libraries were screened using a conventional protein-protein interaction (PPI) assay that was modified. Instead of the common “down” screen in search of PPI-inhibitory small molecules, the assay described herein was set up with KRAS and KEAP1 as a non-interacting pair and an “up” screen was performed looking for compounds that yielded a positive PPI signal. The goal was to identify any small molecule that shows detectable activity in inducing KRAS-KEAP1 interaction (FIG. 2). This type of PPI up-screen has never been systematically tested before. False positive hits associated with gain of signal in up-screens are lower than that in down-screens. KRAS and KEAP1 could present their surface to each other in an infinite number of ways, and a large number of chemicals might have the chance to complement one of these imperfect interactions to promote ternary complex formation.

High-throughput Screen Assay

Primary Screen Assay

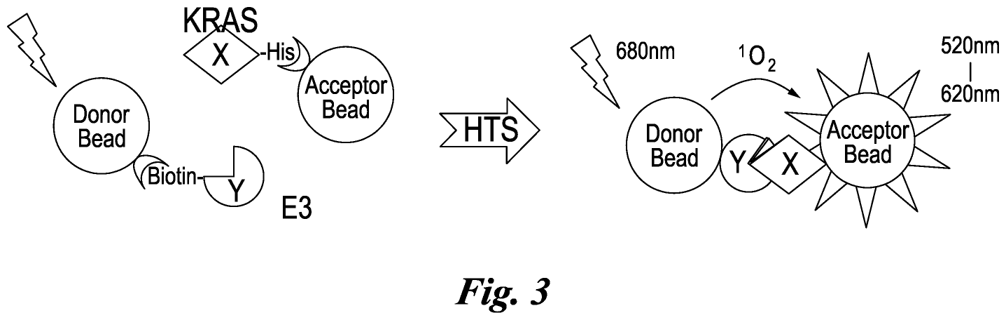

AlphaScreen (Perkin Elmer, Inc., Waltham Wash.) is a bead-based, non-radioactive Amplified Luminescent Proximity Homogeneous Assay. The assay system consists of a donor and an acceptor bead, whose proximity induced by biological interactions will trigger a cascade of chemical reactions that produces a greatly amplified signal. The donor beads used were streptavidin donor beads (PE #6760002s) and the acceptor beads used were Anti-6×His Acceptor beads (PE #AL128C). On laser excitation, a photosensitizer in the donor bead converts ambient oxygen to a more excited singlet state. The singlet state oxygen molecules diffuse across to react with a thioxene derivative in the acceptor bead and generate chemiluminescence at 370 nm that further activates fluorophores contained in the same bead. The fluorophores subsequently emit light at 520-620 nm. AlphaScreen was chosen for its ultrahigh sensitivity suitable for detecting a wide range of affinity (pM to mM), its adaptability for miniaturization, and its potential for multiplexing. In this PPI assay, purified biotinylated KEAP1 kelch repeat domain was immobilized on the streptavidin donor beads, and the 8×His-tagged full-length S^{G12D} mutant protein was immobilized on the anti-His acceptor beads (FIG. 3). Because the two proteins do not normally interact with each other, a mixture of the two beads did not yield any Alpha signal.

The assay was conducted in a 384 well white plate at room temperature. Test solutions contained 25 mM HEPES, pH 7.4, 100 mM NaCl, 0.1% Tween 20, 0.05% BSA, and 1 mM TCEP. 10 μL each of the KEAP1 and KRAS proteins and the test compound were mixed in each well. 10 μL of acceptor beads were added and the mixture was incubated in the dark. Finally, 10 μL of donor beads were added in the mixture was incubated, followed by reading of the plates. The assay can easily tolerate up to 4% of DMSO. Using mean+3× standard deviation (SD) as the hit cut-off, a two-plate pilot test showed that the hit rates reached 1.52%. After the assay was validated, a small-scale pilot screen with a 17,774 compounds library was performed. Based on mean+3×SD cut off, the pilot screen yielded 575 hits with a hit rate of 3.2%.

Compound Validation

Secondary Screen—Cisbio TR-FRET

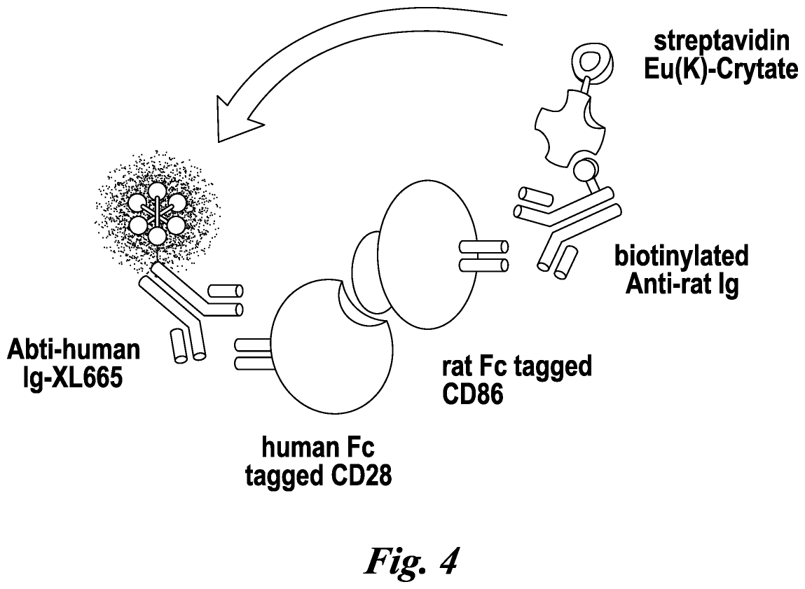

Because the ultra-sensitivity of the AlphaScreen assay, secondary screens orthogonal to AlphaScreen were used to further validate the hit compounds. Cisbio PPI technology, TR-FRET (time-resolved fluorescence resonance energy transfer), was used to confirm the compounds with the highest potency based on dose response studies (FIG. 4).

Out of the compounds that had survived the repeated dose response studies with AlphaScreen and had been tested by TR-FRET, 20 top hits were identified with activities (higher potency with validation by TR-FRET) in promoting KRAS^{G12D}-KEAP1 interaction (FIG. 4).

Secondary Screen—APTLAB

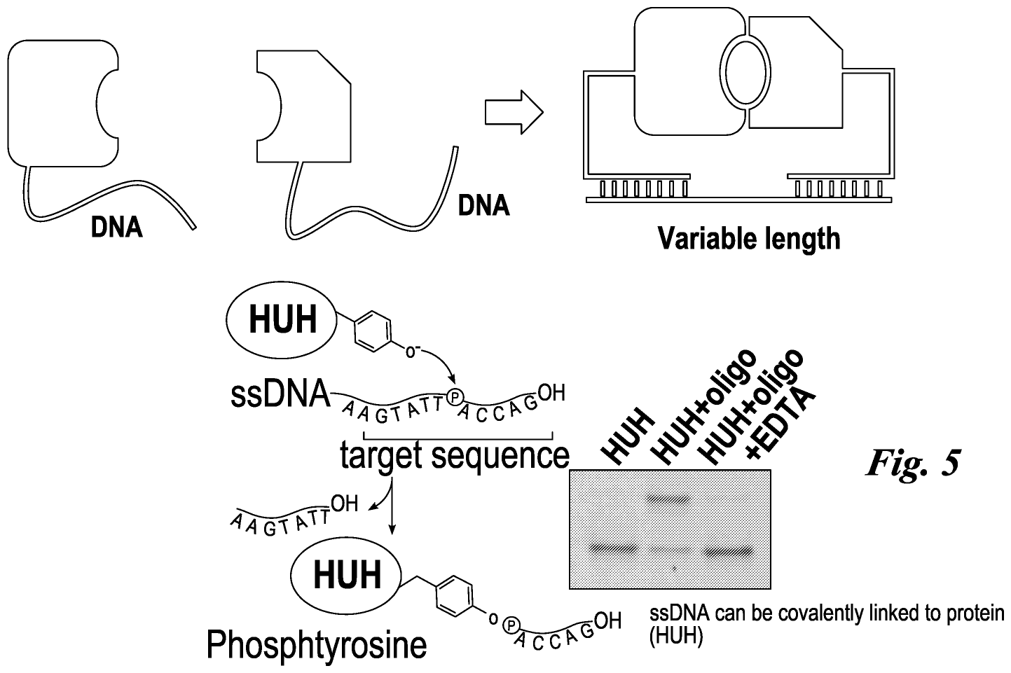

While TR-FRET can be used as an informative second screen for hit validation, its principle is similar to AlphaScreen and relies on beads-based and proximity-induced signals. The hit compounds were further validated with additional methods that are distinct from AlphaScreen and TR-FRET methods. Several conventional methods for detecting PPI, including Octet BioLayer Interferometry (BLI), size exclusion chromatography, and affinity pull down, failed to yield any positive results, suggesting that the activity of the hits might be very low. In other words, even though these compounds might be able to promote KRAS-KEAP1 interaction, the resulting ternary complex might be too unstable to be detected by the conventional methods. To overcome this problem, a new assay was designed (designated herein as APTLAB, adjustable platform for tracking low affinity binding) (FIG. 5), in which weak interactions between complementary DNA strands with variable length was used to enhance the compound-induced low affinity binding between KRAS and KEAP1 and render it detectable by conventional methods.

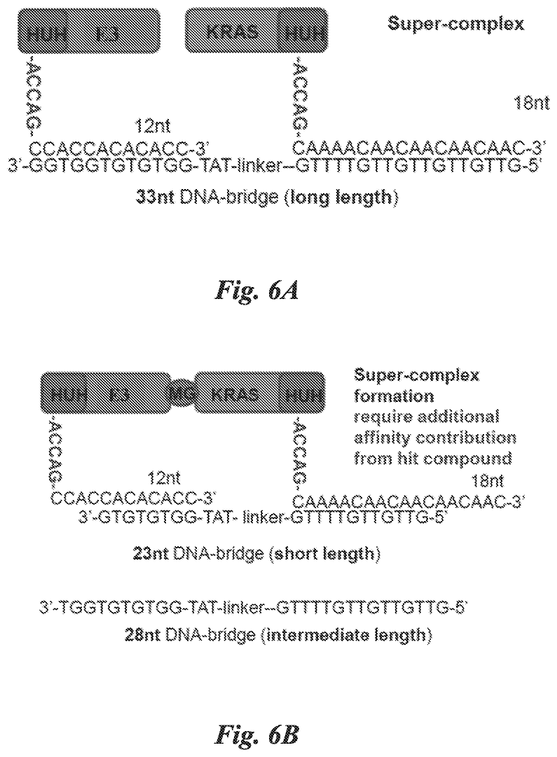

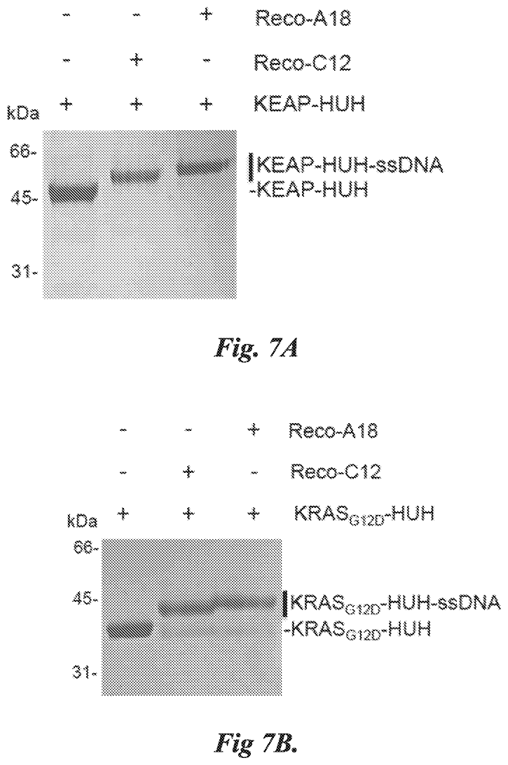

To couple adjustable DNA oligonucleotide interactions with low affinity protein-protein interaction, a protein fold, HUH, which is able to react with a specific single-stranded DNA (ssDNA) oligonucleotide and form a covalently linked protein-DNA conjugate (Lovendahl, K. N., Hayward, A. N. & Gordon, W. R. Sequence-Directed Covalent Protein-DNA Linkages in a Single Step Using HUH-Tags. J Am Chem Soc 139, 7030-7035, doi:10.1021/jacs.7b02572 (2017)), was utilized. HUH protein was fused to the C-terminus of KEAP1 and KRAS^{G12D} individually so that the two chimeric proteins can each form a covalent link with an ssDNA containing the HUH-reactive sequence (FIGS. 5 and 6). The purified HUH fusion proteins were adjusted to 100 μM in a buffer containing 20 mM HEPES (pH 7.5), 50 mM NaCl, 0.5 mM TCEP, 1 mM MgCl₂ and 1 mM MnCl₂. The ssDNA oligonulceotides were added to a final concentration of 120 μM. After incubation at room temperature for 1 hour, the reaction sample was run on an SDS-PAGE gel side by side with a negative control to check the conjugation efficiency. DNA conjugated HUH fusion protein run slower on SDS-PAGE gel.

Beside the HUH-reactive sequence, the two ssDNA oligonucleotides linked to the two chimeric proteins also each contain an additional sequence, which is complementary to one half of an ssDNA bridge oligonucleotide (FIG. 6). When the bridge ssDNA anneals with the two protein-linked oligonucleotides, it will physically bring KRAS and KEAP1 together in a single complex. By varying the length of the bridge ssDNA, the affinity of the DNA-DNA interaction will change. When the bridge oligonucleotide is long enough, the two chimera proteins will stably associate with each other through their DNA parts. Formation of the resulting complex is detectable by a conventional method, such as Octet BLI. When the length of the bridge ssDNA is gradually shortened, the affinity of DNA-DNA interaction will be weakened and become non-detectable. By titrating the length of the bridge oligonucleotide, an inflection point will be observed at a certain length. At this inflection point, additional weak interactions between the two chimera proteins have the potential to enhance the stability of the complex and render it detectable by a conventional method. The rationale behind this approach hinges on the non-linear relationship between binding energy changes and binding affinity difference.

ΔΔG=−RT ln(K_{d1}/K_{d2}) (1)

Based on equation (1), introducing a small amount of additional binding energy can exponentially enhance the affinity of two interacting partners. Overall, APTLAB is designed to detect weak PPI that is augmented by another weak interaction using conventional methods suitable for measuring strong interactions.

To implement APTLAB, a HUH-fused biotinylated KEAP1 and His-KRAS^{G12D} were prepared and performed ssDNA and HUH conjugation assay, in which two ssDNA oligonucleotides were individually linked to biotin-KEAP1 (FIG. 6A) and His-KRAS^{G12D} (FIG. 6B) fused with HUH. ssDNA Reco-C12 linked Biotin-KEAP1-HUH, named KEAP1-ssDNA12, and ssDNA Reco-A18 linked His-KRAS^{G12D}-HUH (KRAS^{G12D}-ssDNA18) were chosen for the following APT-LAB test.

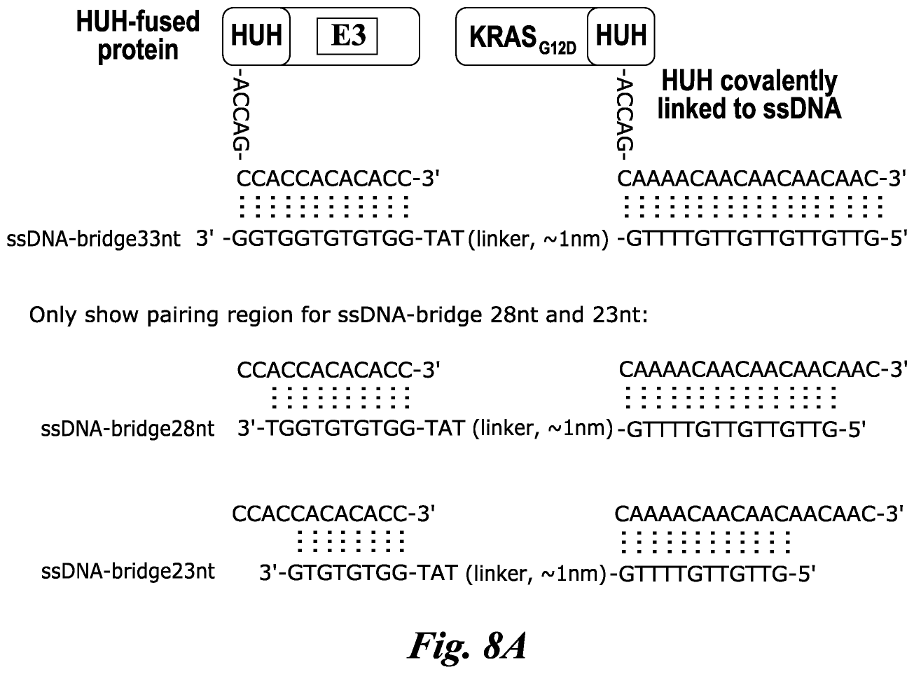

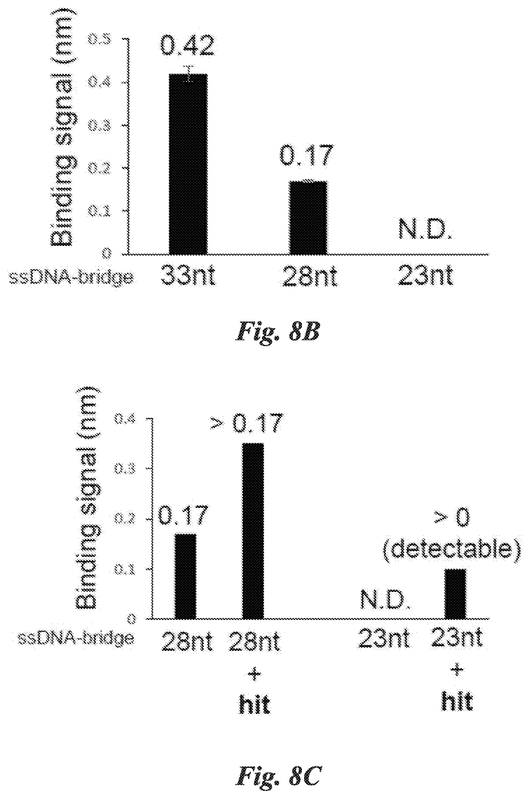

ssDNA bridge oligonucleotides were synthesized with different lengths with 33, 28 and 23 nucleotides (nt) (FIG. 8A) and tested their ability to promote complex formation between the two ssDNA-protein conjugates as monitored by BLI. The longest ssDNA-bridge33nt promoted robust complex formation between KEAP1-ssDNA12 and KRAS^{G12D}-ssDNA18 and yielded a high BLI signal (FIG. 8B). By contrast, the binding signal for ssDNA-bridge28nt is significantly lower, while the interaction mediated by the shortest bridge oligonucleotide ssDNA-bridge23nt was too weak to detect. Thus, ssDNA-bridge-28nt was determined to be the inflection point bridge oligonucleotide, potentially suitable for testing the weak binding between KEAP1 and KRAS induced by the hit compounds.

Compounds were dissolved in DMSO. The ssDNAs used in this research were listed in Table 1. ssDNAs were carefully designed to avoid hairpins. For DNA-DNA pairing region, Reco-C12 applied 33.3% A and 66.7% C, by contrast, Reco-A18 applied 33.3% C and 66.7% A, this difference made ssDNA-bridge bind Reco-C12 and Reco-A18 at the desired direction. The method of in vitro conjugation of ssDNA and HUH was reported previously (Lovendahl, K. N., Hayward, A. N. & Gordon, W. R. Sequence-Directed Covalent Protein-DNA Linkages in a Single Step Using HUH-Tags. J Am Chem Soc 139, 7030-7035, doi:10.1021/jacs.7b02572 (2017)). After conjugation, ssDNA labeled proteins were further purified to remove free ssDNA and unlabeled proteins. KEAP1-ssDNA12 (ssDNA Reco-C12 linked Biotin-KEAP1-HUH) and KRAS^{G12D}-ssDNA18 (ssDNA Reco-A18 linked His-KRAS^{G12D}-HUH) were used for the following binding assay detected by Octet.

Interaction of KEAP1-ssDNA12 (Biotin-tagged) and KRAS^{G12D}-ssDNA18 in the presence of ssDNA-bridge without/with MG was measured using the Octet Red 96 (ForteBio, Pall Life Sciences) following the manufacturer's procedures. First, the streptavidin coated optical probes were hydrated in buffer. Second, the probes were loaded with 50 nM KEAP1-ssDNA12 to optical signal ˜1.6 nM (most streptavidin bound KEAP1-ssDNA12). Third, probes dipped into 500 nM biocytin to occupy remaining free streptavidin on the probes. Forth, probes went into buffer for baseline. Fifth, probes inserted into sample wells (mixture of 1 uM KRAS^{G12D}-ssDNA18 and 82.5 nM ssDNA-bridge without/with compounds) to detect association of KRAS^{G12D}-ssDNA18 with KEAP1-ssDNA12, optical signal increase indicated binding happened and was treated as binding signal. Sixth, probes immersed into buffer to monitor the dissociation of bound KRAS^{G12D}-ssDNA18. The reaction was carried out in black 96 well plates maintained at 30° C. and the reaction volume was 200 μL in each well. The assay buffer contained 25 mM HEPES, pH 7.4, 100 mM NaCl, 0.1% Tween 20 with 0.05% BSA and 1 mM TCEP added freshly.

TABLE 1
ssDNA

| | Sequence (note: lower case |
|---|---|
| ssDNA | sequence is recognized by HUH.) |
| Reco-C12 | 5′-aagtattaccagCCACCACACACC-3′ |
| (SEQ ID NO: 2) | |
| Reco-A18 | 5′-aagtattaccagCAAAACAACAAC |
| (SEQ ID NO: 3) | AACAAC-3′ |
| ssDNA- | 5′-GTTGTTGTTGTTGTTTTGTATGGT |
| bridge33 nt | GTGTGGTGG-3′ |
| (SEQ ID NO: 4) | |
| ssDNA- | 5′-GTTGTTGTTGTTTTGTATGGTGTG |
| bridge28 nt | TGGT-3′ |
| (SEQ ID NO: 5) | |
| ssDNA- | 5′-GTTGTTGTTTTGTATGGTGTGTG-3′ |
| bridge23 nt | |
| (SEQ ID NO: 6) | |

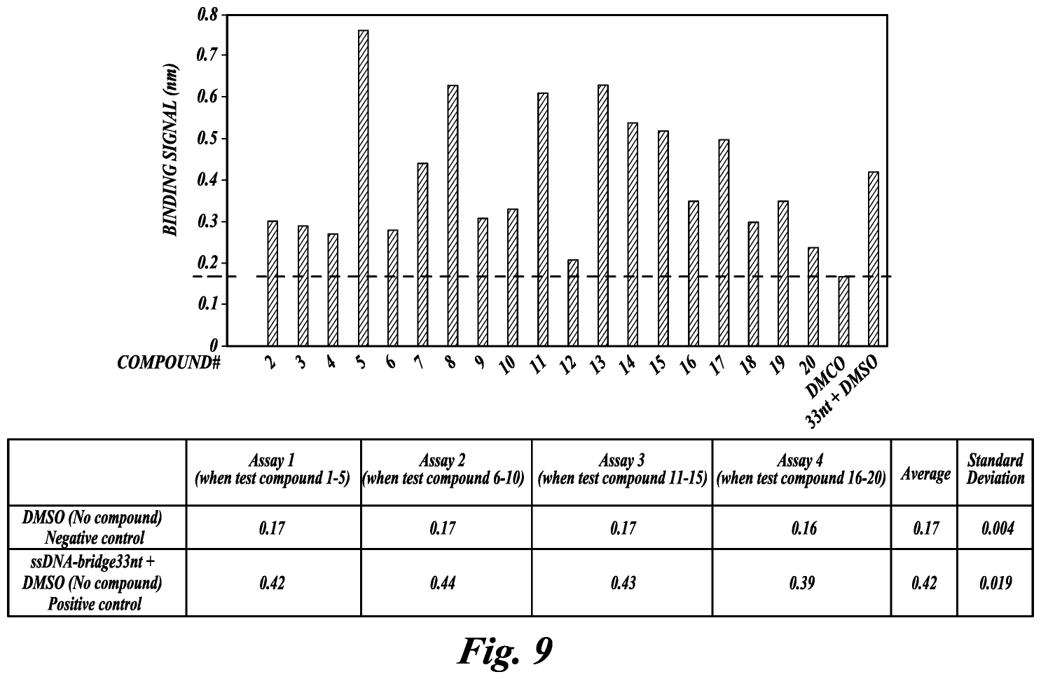

Under this experimental setting, one of the primary screen hit compounds was tested. In comparison to DMSO only control, addition of the hit compound significantly increased the BLI signal with KEAP1-ssDNA12 and KRAS^{G12D}-ssDNA18 (FIG. 8C). The effect was also dose-dependent and relied on the presence of both KEAP1-ssDNA12 and KRAS^{G12D}-ssDNA18. These results strongly suggested that the hit compound had an activity of promoting KEAP1-ssDNA12 and KRAS^{G12D}-ssDNA18 interaction, most likely by binding at the interface between the two proteins. Using a similar approach, the activities of the majority of the 20 hit compounds identified in the TR-FRET secondary screen were validated (FIG. 9).

Secondary Screen—Split Luciferase Assay

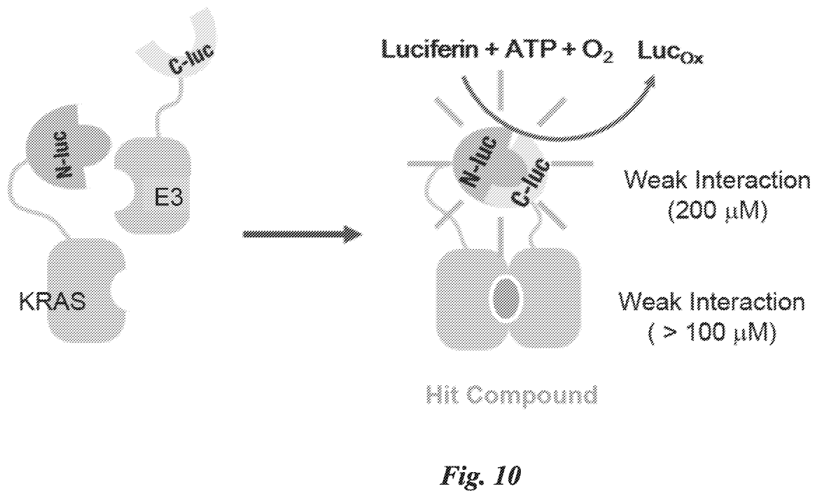

In parallel to the development of APTLAB, an alternative secondary screen assay was developed using the same principle of detecting weak interactions augmented by additional weak interactions. A split luciferase assay was developed based on an assay (FIG. 10) (Dixon, A. S. et al. NanoLuc Complementation Reporter Optimized for Accurate Measurement of Protein Interactions in Cells. ACS Chem Biol 11, 400-408, doi:10.1021/acschembio.5b00753 (2016)) that is a small and stable luciferase, which can utilize furimazine to produce bright luminescence in the presence of molecular oxygen. The enzyme (PDB ID: SIBO) is split into two parts, the Large BiT (LBiT; 159 amino acids, 17.6 kDa) and Small BiT (SBiT; 11 amino acids), which can be fused to two proteins of interest. If there is a protein-protein interaction that can bring these two fusion proteins into close proximity, LBiT and SBiT is able form a functional Nanoluc enzyme to produce bright luminescent signal. Because the intrinsic affinity between LBiT and SBiT is very weak (K_{d}=190 μM), this technology is suitable for detecting weak protein-protein interaction, such as the interactions between KEAP1 and KRAS^{G12D} induced by molecule glue compounds.

To implement this design, SBiT and LBiT were fused to the N-terminus of KRAS^{G12D} and the C-terminus of KEAP1, respectively. Because KRAS^{G12D} and KEAP1 do not naturally interact with each other, a mixture of the two chimera proteins and luciferin produced very low luminescence signal.

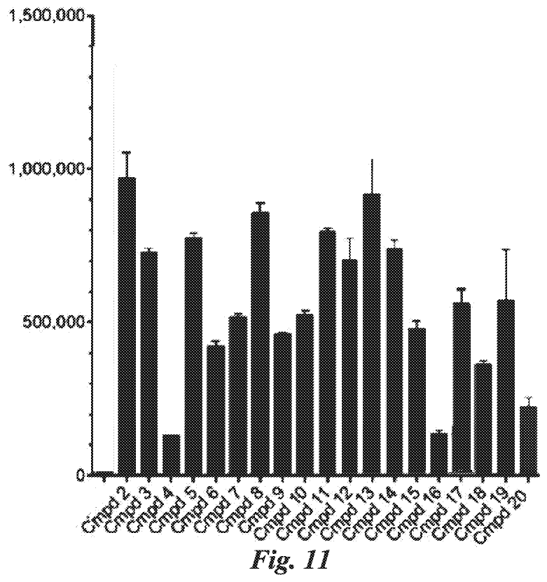

The split luciferase assay was performed following the general procedures described for the NanoLuc Binary Technology developed by Promega. The SBiT-KRASG12D and KEAP1-LBiT fusion proteins were mixed at a final concentration of 2.5 nM in a 50 μL buffer containing 20 mM HEPES, pH 7.5, 100 mM NaCl, and 0.1% BSA-FAF and incubated for 1 hr. 50 μL of the same reaction buffer was mixed with 2 μL of luciferin stock solution purchased from Promega, which was then added to the protein mixture solution in a 96-well white plate, which was subsequently shaken for 2 minutes. With the plate covered by a plastic sheet, the reaction time course was monitored with a Perkin Elmer Enspire 2300 Multiplate Reader at 4 minutes after luciferin mixing. Using this assay, the majority of the 20 hit compounds identified in the TR-FRET secondary screen were validated (FIG. 11).

Protein Preparation Methods

Cloning

All constructs for different assays were designed based on the structures of the human KEAP1 kelch domain (residues 320-612, UniProtKB-Q14145, hereafter referred to as KEAP1, PDB ID: 2FLU) and full length human KRAS4B (UniProtKB-P01116-2) with a site mutation G12D (hereafter referred to as KRAS^{G12D}, PDB ID: 4DSN).

All protein-coding DNA sequences were amplified by PCR and cloned into one set of ligation-in-dependent (LIC) expression vectors modified from pET15 and pET41. Both 8×His-(GS)₄-KRAS^{G12D} and 8×His-(GS)₄-KRAS^{G12D}-(GS)₈-HUH were inserted into pET41 LIC vector. Other constructs were cloned into a pET15 LIC vector bearing an N-terminal 6-Histidine tag. An N-terminal Maltose Binding Protein (MBP) fusion tag was used to improve the yield of 114-(GS)₈—KRAS^{G12D}.

Protein Expression and Purification

The plasmids were transformed into BL21 (DE3) E. coli host, and cells were grown at 37° C. to an optical density of ˜0.5 at 600 nm and then transferred to 16° C. Then isopropyl-β-d-1-thiogalactopyranoside (IPTG) was added to a final concentration of 0.2 mM when absorbance reached 0.8. Cells were harvested 16 h after induction of IPTG at 16° C.

The cell pellet was resuspended in a buffer containing 20 mM Tris-HCl, pH 8.0, 200 mM NaCl, 0.5 mM tris(2-carboxyethyl)phosphine (TCEP), 20 mM imidazole and 1 mM phenylmethylsulfonyl fluoride (PMSF). After lysed by passing through a microfluidizer, the cell lysate was centrifuged at 20,000 g for 1 hour and the supernatant was loaded onto a Ni-NTA column. The target protein was eluted with 200 mM imidazole in the same buffer.

For KEAP1 constructs, the N-terminal His-tag was cleaved by the tobacco etch virus (TEV) protease. After biotinylation, the KEAP1 proteins were further purified by ion exchange and size-exclusion chromatography. After eluted from Ni-NTA column, KRAS^{G12D} was further purified on a HiTrap-SP column (GE Healthcare). Then a nucleotide exchange assay was performed to obtain homogeneous GTP-bound KRAS^{G12D}, followed by size-exclusion chromatography.

Biotinylation Reaction

AviTag™ technology (Avidity) was used for biotinylation of KEAP1 constructs. AviTag (sequence: GLNDIFEAQKIEWHE (SEQ ID NO: 1)) is a substrate of the E. coli biotin ligase (BirA) enzyme, which conjugates a biotin molecule to the lysine residue. After adjusting the concentration of purified AviTag-fused KEAP1 to 100 μM, ATP, magnesium chloride, purified biotin ligase BirA and D-Biotin were added to the final concentrations of 2 mM, 5 mM, 1 μM and 200 μM, respectively. The sample was incubated at room temperature for 1 hour. Biotinylation efficiency was determined by a streptavidin gel-shift assay: prepared two PCR tubes each containing 1 μL of biotinylated KEAP1 sample and 10 μL of 1×SDS-PAGE buffer; heated both samples at 95° C. for 5 minutes; after the samples cool down to room temperature, add 1 μL of 100 μM streptavidin (IBA-Lifesciences) to one of the samples and incubated at room temperature for 5 minutes; run both samples on an SDS-PAGE gel side by side. The complete disappearance of KEAP1 in the presence of streptavidin indicated the completion of biotinylation reaction.

Nucleotide Exchange

Because of its intrinsic GTPase activity, the majority of purified KRAS were eventually switched to the inactive GDP-bound state. To prepare the protein in its activated GTP-bound form, the bound GDP was replaced by non-hydrolyzable GTP analogs, such as guanosine-5′-[(β, γ)-methylene] triphosphate (GMP-PCP). The concentration of KRAS^{G12D} was adjusted to 100 μM in a buffer containing 40 mM Tris-HCl, pH 7.5, 200 mM (NH₄)₂SO₄, 10 μM ZnCl₂, 5 mM DTT and 1 mM GMPPCP. Alkaline phosphatase conjugated to Sepharose beads (Sigma P-0762) was added to 10 U/ml. After incubation at room temperature for 1 hour with gentle agitation, the reaction was supplemented with 10 mM MgCl₂ and the alkaline phosphatase beads was removed by a brief centrifuge spin. The GTP-bound KRAS^{G12D} was further purified by size-exclusion chromagraphy to remove free nucleotides in a buffer containing 20 mM HEPES (pH 7.5), 50 mM NaCl, 10 mM MgCl₂, 0.5 mM TCEP.

The references provided above are incorporated by reference in their entireties.

While illustrative embodiments have been illustrated and described, it will be appreciated that various changes can be made therein without departing from the spirit and scope of the invention.

1. A method for identifying a ubiquitin ligase agonist, comprising:
(a) contacting a ubiquitin ligase with a candidate agonist and a neo-substrate; and
(b) determining whether the candidate agonist is effective to result in binding the ubiquitin ligase to the neo-substrate, wherein binding of the ubiquitin substrate to the neo-substrate identifies the candidate agonist as a ubiquitin ligase agonist.

2. The method of claim 1, wherein binding of the ubiquitin ligase to the neo-substrate provides a complex comprising the ubiquitin ligase, the agonist, and the neo-substrate.

3. The method of claim 1, wherein determining whether the candidate agonist is effective to result in binding the ubiquitin ligase to the neo-substrate comprises observing a signal generated by the binding.

4. The method of claim 1, wherein the ubiquitin ligase further comprises a first reporting agent and the substrate further comprises a second reporting agent, wherein upon binding of the ubiquitin ligase to the neo-substrate, the first reporting agent acts with the second reporting agent to generate a signal.

5. The method of claim 4, wherein the ubiquitin ligase comprising the first reporting agent is a donor bead configured to generate a reactive oxygen species when in the excited state, and the neo-substrate comprising the second reporting agent is an acceptor bead configured to generate light in the presence of a reactive oxygen species upon binding of the ubiquitin ligase to the neo-substrate.

6. The method of claim 5, wherein the donor bead comprises a sensitizer configured to generate the reactive oxygen species when the sensitizer is in an excited state.

7. The method of claim 6, wherein the sensitizer is a photosensitizer configured to generate the reactive oxygen species when the sensitizer is illuminated with stimulation electromagnetic radiation.

8. The method of claim 6, wherein the photosensitizer is a phthalocyanine.

9. The method of claim 5, wherein the acceptor bead comprises a luminescent compound configured to generate luminescent light when the luminescent compound is in proximity to a reactive oxygen species.

10. The method of claim 9, wherein the luminescent compound is selected from the group consisting of thioxene, anthracene, rubrenein, and combinations thereof.

11. The method of claim 5, wherein the reactive oxygen species is singlet oxygen.

12. The method of claim 1, wherein determining whether the candidate agonist is effective to result in binding the ubiquitin ligase to the neo-substrate comprises observing a gain of a signal by the binding.

13. The method of claim 1, wherein the ubiquitin ligase further comprises a first reporting agent and the neo-substrate further comprises a second reporting agent, wherein upon binding of the ubiquitin ligase to the neo-substrate, the first reporting agent acts with the second reporting agent resulting in a gain of a signal.

14. The method of claim 1, wherein the ubiquitin ligase is a member of the cullin-RING superfamily of multi-subunit E3 ubiquitin ligases, a member of RING-type E3 ligases with a substrate binding domain, or a member of HECT-type E3 ligases with a substrate binding domain.

15. The method of claim 1, wherein the ubiquitin ligase is KEAP1.

16. The method of claim 1, wherein the neo-substrate is KRAS or KRAS mutant.


SEQUENCE LISTING (6 sequences)

SEQ ID NO: 1 - protein, 15 aa, Artificial Sequence
  Synthetic
GLNDIFEAQKIEWHE

SEQ ID NO: 2 - DNA, 24 bp, Artificial Sequence
  Synthetic
aagtattaccagccaccacacacc

SEQ ID NO: 3 - DNA, 30 bp, Artificial Sequence
  Synthetic
aagtattaccagcaaaacaacaacaacaac

SEQ ID NO: 4 - DNA, 33 bp, Artificial Sequence
  Synthetic
gttgttgttgttgttttgtatggtgtgtggtgg

SEQ ID NO: 5 - DNA, 28 bp, Artificial Sequence
  Synthetic
gttgttgttgttttgtatggtgtgtggt

SEQ ID NO: 6 - DNA, 23 bp, Artificial Sequence
  Synthetic
gttgttgttttgtatggtgtgtg